(19) United States
(12) Patent Application Publication
(10) Pub. No.: US 2025/0034636 A1
(43) Pub. Date: Jan. 30, 2025

(54) SINGLE-CELL CULTURE AND SEQUENCING WITH LIPID-MODIFIED OLIGOS

(71) Applicants: CZ BIOHUB SF, LLC, San Francisco, CA (US); THE REGENTS OF THE UNIVERSITY OF CALIFORNIA, Oakland, CA (US)

(72) Inventors: Kai-Chun Chang, Oakland, CA (US); Adam Abate, Oakland, CA (US); Sixuan Pan, Oakland, CA (US)

(21) Appl. No.: 18/717,006

(22) PCT Filed: Dec. 8, 2022
(86) PCT No.: PCT/US22/52218

§ 371 Date: Jun. 6, 2024

Related U.S. Application Data
(60) Provisional application No. 63/287,249, filed on Dec. 8, 2021.

(51) Int. Cl. C12Q 1/6869; C12Q 1/6806

(52) U.S. Cl. CPC C12Q 1/6869; C12Q 1/6806

(57) ABSTRACT

Existing spatial transcriptomics technologies require dead tissues and are not compatible with live cell cultures. The present disclosure provides materials and methods for sequencing a single cell from a cell culture sample and obtaining morphologic or phenotypic measurements and information by combining sequencing approaches and spatial hashing (e.g., barcoding) at a single cell level.

### CROSS-REFERENCE TO RELATED APPLICATIONS

The present application claims priority to U.S. Provisional Patent Application No. 63/287,249, filed on Dec. 8, 2021, their entirety of which are incorporated by reference herein.

### STATEMENT REGARDING FEDERALLY SPONSORED RESEARCH

This invention was made with government support under grants U01 AI129206, R01 AI149699, and R41 GM136037 awarded by The National Institutes of Health. The government has certain rights in the invention.

### FIELD

The present disclosure relates generally to methods for sequencing a single cell from a cell culture sample and obtaining morphologic or phenotypic measurements and information by combining sequencing approaches and spatial hashing (e.g., barcoding) at a single cell level.

### BACKGROUND

Single-cell sequencing has demonstrated unappreciated cellular diversity in many ostensibly homogeneous systems over the past few years and led to an ongoing scientific revolution in cell biology (Klein, A. M., et al., (2015) Cell, 161, 1187-1201.; Macosko, E. Z., et al. (2015) Cell, 161, 1202-1214.; Zheng, G. X. Y., et al. (2016) Nat. Commun., 8, 065912.; Pellegrino, M., et al. (2018) Genome Res., 28, 1345-1352.). Droplet microfluidics and deep sequencing are the major drivers behind this revolution (Klein et al., supra).

Existing spatial transcriptomics technologies require dead tissues and are not compatible with live cell cultures. Current single-cell sequencing technologies do not include spatial indexes that allow users to map single-cell RNAseq data to cell morphology. To integrate single cell morphology and gene expression, researchers often resort to manually picking cells of interest using well-based methods, which greatly limits the throughput. Common spatial transcriptomics products are optimized for frozen or fixated tissues and require specialized and expensive substrates that are not compatible with long-term cell culture and high-resolution imaging. In situ sequencing and profiling methods sacrifice detection efficiency and/or the number of genes being probed. Thus, there remains a need for a method of obtaining morphologic or phenotypic measurements and information linked with sequence information at single-cell precision.

### SUMMARY OF THE INVENTION

One embodiment of the present disclosure provides a method of determining the sequence of at least one nucleic acid from a single cell, said method comprising the steps of: (a) preparing a cell culture substrate comprising a plurality of single cells or micro-colonies produced from a single cell; (b) preparing a labelling substrate, wherein said labelling substrate comprises oligonucleotides comprising spatial barcodes; (c) labelling single cells or micro-colonies of (a) with labelling substrates of (b) or with liquid dispensers that directly print spatial barcodes to (a); (d) preparing a suspension comprising labeled single cells or micro-colonies; and (e) determining the sequence of at least one nucleic acid from at least one labeled, single cell from the suspension.

In one embodiment, the cell culture substrate comprises a surface or a series of wells capable of compartmentalizing micro-colonies or single cells. In another embodiment, micro-colonies or single cells are deposited onto a surface. In still another embodiment, micro-colonies or single cells are deposited into nano-wells.

The present disclosure also provides, in some embodiments, an aforementioned method wherein said method further comprises the step of determining at least one phenotypic or morphologic measurement of the single cells after step (a) and prior to step (c). In another embodiment, said measurement is selected from the group consisting of imaging, traction force microscopy (TFM), atomic force microscopy (AFM), mass spectrometry (MS), and enzyme assays.

In still other embodiments, an aforementioned method is provided wherein the labelling substrate comprises a lipid-modified oligonucleotide (LMO). In other embodiments, the labelling substrate is embedded within a gel scaffold. In still other embodiments, the labelling substrate is deposited onto a surface. In one embodiment, the surface is selected from the group consisting of an array, a slide, a PDMS slab, a layer of patterned or unpatterned hydrogels, a SU8-coated slide, and a membrane sheet. In still other embodiments, the labelling in step (c) is carried out under conditions that allow labelling substrates to contact single cells or micro-colonies. In another embodiment, said conditions comprise inducing release of labelling substrates from the surface. In another embodiment, the labelling oligos are printed directly to cells with a liquid dispenser. In still other embodiments, the suspension of (d) is prepared by disassociating and collecting the labeled single cell or micro-colonies. In yet other embodiments, the at least one phenotypic or morphologic measurement is correlated with sequencing information of step (e).

### BRIEF DESCRIPTION OF THE DRAWINGS

FIG. 1 shows cultured iPS-derived neurons in nanowell array. Cell body is stained with calcein green. Nucleus is stained with DRAQ5.

FIG. 2 shows a workflow of spatial hashing and preliminary data of oligo transfer from the patterned hashing substrate. FIG. 2a) LMOs (anchor and co-anchor) are sequentially pipetted onto cells for insertion. Spatial hashes are then stamped onto cells for transfer and labeling. Here we replace the spatial hashes with fluorescent dummy oligos for visualization to facilitate method development. FIG. 2b) A hashing substrate printed with checkerboard pattern of dummy hashes (Cy5 hash) is prepared and then stamped onto calcein green-stained cells. FIG. 2c) After stamping, the nanowells that are filled with Cy5 hashes are highlighted with circles. Note that cells labeled with Cy5 hashes show strong red fluorescence (cells in the highlighted nanowells), while cells not in direct contact with Cy5 hashes do not (cells in the center nanowell), indicating limited crosstalk of spatial hashes between nanowells.

FIG. 3 shows a library prep of spatially hashed cells cultured in nanowell array. As a pilot experiment, human (HEK293) and mouse (3T3) cells are cultured, hashed and then sequenced. FIG. 3a) The spatial hash fraction after library prep. FIG. 3b) The cDNA fraction of cells. FIG. 3c) Two-species barnyard plot of the cDNA fraction. The transcripts from either species are well separated from each other, indicating rare pre-lysis and co-encapsulation events of cells (only 10 multiplets were detected in over 800 cells).

FIG. 4 shows a workflow for spatial hashing of live cell monolayers. FIG. 4a) Arrayed LMOs containing spatial barcode are stamped onto live cell monolayer sample slide. FIG. 4b) LMOs are released into the cells in the stamped area. The spatially hashed cells can then be resuspended for single-cell sequencing. FIG. 4c) tSNE plot with colors indicating clusters, which correspond to spatial position of cells from the original sample slide.

FIG. 5 shows patterned cells, corresponding traction stress maps (figure adapted from Muncie, J. M., et al. Developmental Cell. 2020. 55 (6), 679-694), and, for illustration, schematic tSNE plot with colors indicating expected clustering.

FIG. 6 shows cells cultured on a flat slide without nanowells are stained with calcein green, and then stamped with checkerboard-patterned Cy5 hash. Only cells adjacent or in direct contact with the printed area show red fluorescence, indicating limited crosstalk across the slide even without nanowells.

FIG. 7 shows a workflow for co-profiling of metabolites and transcriptome of each isogenic microcolony. FIG. 7a) Yeast microcolonies are cultured in droplets and then sorted and printed on to MALDI substrate with PDM (adapted from Xu, L., et al. Nat Commun 2021.12, 6803). FIG. 7b) After MALDI, spatial hashes are inserted into yeast cells by stamping or printing. The microcolonies are gelled and then resuspended for “single colony” sequencing.

FIG. 8 shows the results of an investigation as to whether nucleic acids, such as DNA and RNA, can be recovered after MALDI, human (HEK293) and mouse (3T3) cells are printed onto MALDI substrate with PDM. The two-species barnyard plot shows that a significant fraction of RNA can still be recovered after MALDI, albeit with a lower quality in the pilot experiment.

FIG. 9 shows recovering yeast microcolonies after MALDI for RNAseq. FIG. 9a) Printed microcolonies by PDM. FIG. 9b) Application of MALDI matrix by airbrushing. FIG. 9c) After MALDI, the matrix is washed away by 70% ice-cold ethanol to prevent interfering with downstream RNA recovery. FIG. 9d) Yeast microcolonies are encapsulated with hydrogels and labeled with LMO and Cy5 hash (bright spots). FIG. 9e) Resuspending the gelled microcolonies. The red fluorescence remains on the yeast, indicating stable LMO insertion and spatial hashing (bright dots). FIG. 9f) Co-encapsulation of gelled microcolonies (bright spots highlighted with arrows) and sequencing beads (small spheres in droplets).

FIG. 10 shows the results from linking metabolome and transcriptome for metabolic engineering. FIG. 10a) Clustering of yeast microcolonies based on mass spec peaks from MALDI (figure adapted from Xu, L., et al. Nat Commun. 2021. 12, 6803). FIG. 10b) Expected integration of gene expression and metabolite production.

### DETAILED DESCRIPTION

The present disclosure provides, in various embodiments described herein, methods to meet the aforementioned need in the art. In particular, the present disclosure enables high-throughput integration of single-cell long-term culture, imaging, and RNAseq without specialized substrates.

In certain embodiments, the present disclosure provides lipid-modified oligonucleotides (LMO) and a liquid dispenser, for example Scienion's sciFLEXARRAYER S3 (such as that used in Lee Y, et al., (2021) Sci Adv.), Genetix's QArray2 (such as that used in Srivatsan S R, et al., (2021) Science and ArrayJet's ArrayJet Spider system (such as that used in Vickovic, et al., (2016) Nat Commun.) to spatially label cultured, single cells grown on any surface. The LMOs are dispensed on a dry and flat surface (e.g., a Polydimethylsiloxane (PDMS) slab) using the liquid dispenser, and the surface is subsequently stamped onto the cell culture of interest as described herein. Alternatively, the spatial oligos are directly printed to cells with liquid dispensers. The cells are re-suspended into solution, for example by trypsinization, and then subjected to standard a single-cell sequencing platform. With this invention, imaging-based phenotypic observation on live culture cells can be unambiguously linked with transcriptome information with a simple stamping step.

Single cell sequencing methods and commercially available platforms include, without limitation, 10X Genomics' Chromium, Drop-seq, inDrop, Fluent BioSciences' PIPseq 3′ Single Cell RNA Kit, Mission Bio's Tapestri, Fluidigm's C1, Bio-Rad's ddSEQ, and Takara's ICELL8.

In various embodiments, the present disclosure enables cells cultured on common dishes or substrates to be spatially labeled and then sequenced with common RNAseq platforms. The integration of cell morphology and gene expression measurements based on the present disclosure can further enable discovery of oncogenic cells based on imaging or selection of engineered cell lines (e.g. CAR-T) that have desired cell-cell interaction properties that, for example, kill cancer cells.

As described herein, in one embodiment of the present disclosure a method of determining the sequence of at least one nucleic acid from a single cell, said method comprising the steps of: (a) preparing a cell culture substrate comprising a plurality of single cells or micro-colonies produced from a single cell; (b) preparing a labelling substrate, wherein said labelling substrate comprises oligonucleotides comprising spatial barcodes; (c) labelling single cells or micro-colonies of (a) with labelling substrates of (b) or with liquid dispensers that directly print spatial barcodes to (a); (d) preparing a suspension comprising labeled single cells or micro-colonies; and (e) determining the sequence of at least one nucleic acid from at least one labeled, single cell from the suspension.

As used herein, the term “cell culture substrate” means any surface upon which cells are loaded. A cell culture substrate can be silica, a polymeric material, glass, beads, chips, slides, or a membrane. A cell culture substrate may meet the physiological needs for cells in various aspects, including but not limited to cell survival, growth, division, differentiation, and attachment. A cell substrate may meet other requirements for the desired methods of phenotypic analysis, such as confocal, MALDI and TFM.

As used herein, the term “micro-colony” refers to a group of closely packed biological particles, such as cells, including yeast cells and bacteria cells. A micro-colony typically has a diameter between 1 and 1000 micrometers. Biological particles within a given micro-colony may possess similar or identical properties, such as genetic composition or being exposed to the same drugs, chemicals or biological reagents.

As used herein, the term “liquid dispenser” refers to the machinery that deposits biological materials, such as nucleic acids, proteins or drugs, to any given surface.

As used herein, the term “labelling substrate” or “hashing substrate” refers to the solid support of a nucleic acid array. The nucleic acids can be transferred to cells by placing such labelling or hashing substrates directly on or above the cells. The labelling or hashing substrate can be silica, a polymeric material, glass, beads, chips, slides, or a membrane.

As used herein, the term “suspension” refers to an aqueous solution of dispersed and suspending biological particles, such as single cells or gel-encased micro-colonies. The suspension is typically created by mixing and agitating the biological particles from a solid support or from another existing suspension.

The term “sample” or “biological sample” encompasses a clinical sample, and also includes cells in culture, cell supernatants, cell lysates, cells, serum, plasma, biological fluid, and tissue samples. As described more fully herein, in various aspects the subject methods may be used to detect a variety of components from such biological samples. Components of interest include, but are not necessarily limited to, cells (e.g., circulating cells and/or circulating tumor cells), viruses, polynucleotides (e.g., DNA and/or RNA), polypeptides (e.g., peptides and/or proteins), and many other components that may be present in a biological sample.

The terms “oligo” and “oligonucleotide” and “polynucleotide” and “nucleic acid” and “target nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 109 nucleotides or larger. Polynucleotides and nucleic acids include RNA, cDNA, genomic DNA. In particular, the polynucleotides and nucleic acids, is used herein to refer to a binding moiety used in the methods described herein and/or as a target of the methods described herein (e.g., a target whose location and sequence is determined by practicing the methods described herein).

The term “oligonucleotide” in some embodiments refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, CA)) according to specifications provided by the manufacturer or they can be the result of restriction enzyme digestion and fractionation.

The term “primer” as used herein refers to a polynucleotide, typically an oligonucleotide, whether occurring naturally, as in an enzyme digest, or whether produced synthetically, which acts as a point of initiation of polynucleotide synthesis when used under conditions in which a primer extension product is synthesized. A primer can be single-stranded or double-stranded.

The term “array” or “nucleic acid array” as used herein refers to a regular organization or grouping of nucleic acids of different sequences immobilized on a solid phase support at known locations. The nucleic acid can be an oligonucleotide, a polynucleotide, DNA, or RNA. The solid phase support can be silica, a polymeric material, glass, beads, chips, slides, or a membrane. The methods of the present invention are useful with both macro- and micro-arrays.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al, Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

“Detecting” or “determining” as used herein generally means identifying the presence of a target, such as a target nucleic acid or protein or biomolecule. In various embodiments, detection signals are produced by the methods described herein, and such detection signals may be optical signals which may include but are not limited to, colorimetric changes, fluorescence, turbidity, and luminescence. Detecting, in still other embodiments, also means quantifying a detection signal, and the quantifiable signal may include, but is not limited to, transcript number, amplicon number, protein number, and number of metabolic molecules. In this way, sequencing or bioanalyzers are employed in certain embodiments.

According to some embodiments of the present disclosure, a lysing reagent is used in the detection methods. Lysing reagents may include, for example chemical lysis, such as SDS, detergents, alkaline, and acid; biological lysis, such as lysis enzymes, viruses, and phages; and physical lysis such as beads beating, grinding, frozen-thaw, and sonication, heating, cutting, and laser or ion beams.

The present disclosure provides methods of detecting a target in a sample, where the target may be, for example, a nucleic acid (RNA, DNA), biomolecules such nucleic acids, genes, proteins or polypeptides or epitopes, as well as biological particles such as cells (bacterial, human, parasite) and viruses. As used herein, “biomolecules” can be nucleic acids themselves or can be other biomolecules that are associated with nucleic acids or comprise nucleic acids such as cells, proteins, or nuclei and the like. The term “substrate” or label substrate” or labelling substrate” includes, without limitation, a slide or an array or, in one embodiment can be the tissue sample itself.

As described herein, the present disclosure provides methods for simultaneous recovery of gene expression and spatial information by nucleic acid sequencing approaches. The methods allow cells to be cultured on any desired substrate that is suitable for morphological and/or other multimodal phenotypic assays, such as imaging, traction force microscopy (TFM), atomic force microscopy (AFM), or mass spectrometry (MS). After the desired multimodal analysis, a hash substrate is then stamped onto the cells, where the hashes can directly insert into the cell surface through lipid moieties or through a LMO adapter with binding sequences. The cells can thus be hashed in situ without affecting cell viability. The spatially hashed cells are dissociated to prepare a particulate suspension, and the suspension can be further processed or sorted based on cell characteristics or original pixel locations, if desired. In some embodiments, the suspension is then barcoded and subjected to single-cell barcode sequencing, thereby providing sequence information that can be used to infer the original locations and integrate prior multimodal datasets of the cells based on combinations of barcode and hash sequences.

As will be appreciated by those in the art, numerous workflows are possible and contemplated herein.

### Preparing Cell Culture

Cells can be grown separately in nano-well arrays or as a monolayer on bare surfaces until the morphological and other phenotypic characteristics are properly developed. The culture substrate may be modified with substances needed for cell culture and desired assays. Cells can also be cultured in droplets to form microcolonies and then printed down to the substrate. The cultured cells are then processed for downstream in situ analysis, such as antibody binding, dye staining, nucleic acid probe binding, drug treatment or MS matrix application. Common in situ assays include imaging, AFM, TFM, MS, etc. Single cells may be cultured in separate nano-wells to enable high-throughput drug screening. For example, in one embodiment, after single cells are loaded into the nano-well array, a drug array is stamped onto the nano-well array and incubated for parallel drug treatment. The cells can then be stained and imaged for morphological and phenotypic analysis. Multiple cells can be cultured within each nano-well. For example, in one embodiment, multiple cell types are loaded into each nano-well. Since each nano-well is labeled with a unique spatial hash or hash combinations, and that each single cell is barcoded separately during preparation for sequencing, gene expression patterns arisen from cell-cell interactions can be analyzed within each nano-well by combining the information from spatial hashes and cell barcodes. This method can also be applied to live cell monolayers, tissues or organoids. For example, in one embodiment, a deformable hashing substrate may be stamped on the brain of an anesthetized mouse in situ, and then the cells are recovered for sequencing. In addition to single cells, this method can be applied to chunks of cells or cell micro-colonies. For example, in one embodiment, cell micro-colonies are gelled on the substrate after multimodal analysis, and then spatially hashed for downstream sequencing.

### Preparing a Hashing Substrate

To provide spatial sequencing, in one embodiment a hashing substrate is micropatterned with spatial hashing oligos at different locations. The hash oligos may be lipid-modified and directly inserted into the cell surface or contain binding sequences to bind existing LMO oligos loaded to cells. Once stamped and released, these hash oligos can spatially hash the cells. The hashing substrate can be a bare surface or an array of nano-wells. In some embodiments, it is desirable to cast gel on the hashing substrate to create a diffusion barrier and/or trap a high amount of materials, such as hash oligos, drugs, nucleic acid probes, proteins and others within the gel mesh. Gelation can be accomplished with various materials, such as polyacrylamide or agarose. Gelation can be initiated using a variety of techniques, such as photo, chemical or thermal initiation. Gels may be micropatterned as compartmentalized gel patches with various methods, such as printing by a commercial liquid handler or light-induced gelation with a photomask. For example, in one embodiment, the gels are printed into nano-wells as reservoirs for materials and then cross-linked with light exposure and photoinitiator. The hash oligos can be deposited on the substrate through a number of techniques, such as printing by a commercial liquid dispenser as described herein or array-based synthesis by photoinitiated chemistries, as are used to make microarrays. Hash oligos may be reversibly fixed on the hashing substrate via cleavable functional groups or via the binding sequences of oligos cross-linked to the hashing substrate. For example, in one embodiment, both the hashing substrate and oligos have functional groups that can be chemically cross-linked via click chemistry. In another embodiment, hash oligos bind to the cleavable oligos cross-linked to the hashing substrate. In either embodiment, after stamping, the hash oligos can be released by temperature melting, photo-mediated cleavage, enzymatic cleavage, pH, or oxidation-reduction reactions. The hash oligos can be micropatterned on the hashing substrate that matches spatial distribution of cells. For example, in one embodiment, cells are grown in patches of different shapes via a patterned culture substrate. The hash oligos on the hashing substrate are micropatterned to maximize resolution within these cell patches or regions of interest, whereas regions with low cell densities are omitted. Materials such as drugs, proteins, dyes, can also be deposited on the hashing substrate globally on a large portion, over local regions, or with micropatterning for various cell treatments. For example, in one embodiment, fluorescent labeling molecules are printed in regions of interest to spatially label the cells for downstream fluorescence-based sorting and isolation.

### Applying the Hashing Substrate and Releasing Hashes

After bringing the hashing substrate to proximity of cells, hash oligos released from the hashing substrate may be directly inserted into the cell surface through lipid moieties. Alternatively, cells may be treated with LMOs before stamping the hashing substrate. The nucleic acid binding sequences on LMOs can then bind to hash oligos released from the hashing substrate. Two or more lipid types of LMOs may be added to co-stabilize the insertion. In some embodiments, it is desirable to gel the cell microcolonies before spatial hashing. Gelation can be accomplished with various materials, such as polyacrylamide or agarose. Gelation can be initiated using a variety of techniques, such as photo, chemical or thermal initiation. Gels may be micropatterned according to cell locations, such as printing by a commercial liquid handler or light-induced gelation with a photomask. After stamping and release of hash oligos, they may form gradient patterns radiating from the deposited positions due to diffusion. Modeling the diffusion gradient detected by imaging and/or sequencing can be used to interpolate higher resolution for cells or gel chunks between the deposited spots of hash oligos.

### Printing Spatial Oligos Directly to Cells

In some embodiments, the spatial oligos are directly brought into close contact by commercial liquid dispenser without the intermediate of the hashing substrate. The cells being labelled can be crosslinked, fixed or gelled before or after LMO insertion and hash printing.

### Dissociating Cells to Create a Suspension

Spatially hashed cells or gel chunks must be dissociated and re-suspended into particulate suspensions to facilitate single-cell sequencing. Hashed cells or gel-encased microcolonies may be physically re-suspended for example by mechanical shaking or pipetting. Hashed cells may be dissociated by enzymatically removing the adhesion molecules between cells and/or between cells and the culture substrate. Reagents may be applied to the cells or gel chunks to further digest cell wall or other extracellular substances to facilitate release of nucleic acids for downstream single-cell sequencing.

### Analyzing and Processing the Cell Suspension

Cell or gel chunk suspensions can be further analyzed or sorted before sequencing. For example, in one embodiment, nuclei, cell surface, cell bodies, or gelled cell chunks in suspension can be labeled with dyes, antibodies, nucleic acid probes, etc. Once labeled, they can be subjected to various analyses, such as flow cytometry, imaging, mass spectrometry/cytometry etc. Desired cells can be sorted and recovered based on their intrinsic characteristic, labels, or spatial hashes. For example, in one embodiment, cells in regions of interest are fluorescently labeled by the hashing substrate and then certain cells are labeled by antibodies in suspension. Certain cell in regions of interest with desirable characteristics can therefore be sorted by flow cytometry for sequencing. Once a desirable cell or gel chunk fraction is obtained, they are partitioned into parallel compartments such as droplets or wells. The compartmentalized parallel processing allows an array of reactions, such as single-cell barcoding, gel removal, rRNA removal, miRNA isolation, DNA fragmentation/tagmentation, etc. For example, in one embodiment, gel is broken down by thermal melting, or reversing crosslinks by application of light or chemicals. The resulting barcoded cells or gel chunks can be subjected to common single-cell sequencing platforms to recover nucleic acid information, such as DNA, RNA, hashes or other labeling oligos.

### Sequencing and Analyzing the Suspension

The processing and sequencing steps described above integrate multimodal datasets, comprising sequencing, imaging, flow cytometry, AFM, TFM, MS, etc data types for each cell or cell micro-colony. These modalities are linked in the datasets by the barcodes, hashes and spatial locations, and can be thus used to inform correlations between these distinct modalities applied to the same cells. For example, in one embodiment, spatial coordinates from imaging are mapped to those inferred from hash sequences. The sequencing readout of each cell can thus be correlated with morphological observations from imaging, for example the localization of certain RNAs from high-resolution images and their expression level from sequencing data. The integration of multimodal datasets may also improve data quality and/or resolution for certain noisy modalities by aggregating the noisier data based on similar cell types, characteristics or clusters inferred from sequencing data or other high-resolution modalities. For example, in one embodiment, noisy spectroscopic data are averaged over cells with similar gene expression patterns to smooth out the spectroscopic noise for the given cell cluster. As mentioned, spatial resolution of hashing may be increased by modeling the diffusion gradient, where concentration gradient of hashes can be measured from sequencing read counts of hash oligos or images of fluorescent hash oligos. The spatial resolution of hashing may be further improved by correlating hash-inferred locations with various images from the same pixel locations. This can follow a recursive process where a graph of all spatial hashes from sequencing data is generated and then aligned to the images. Sequencing data, such as gene expression profiles, for cells with highest positional confidence are correlated with the aligned image readouts at the same pixel locations. Once the relationship between sequencing data and image readouts are established, cells with less positional confidence can search for expected cell characteristics within the hash-inferred region in the image. For example, in one embodiment, cell morphology clusters are correlated with gene expression clusters using cells with highest positional confidence. Next, cells with less positional confidence are assigned with expected morphology based on their gene expression, and then search for cells with the expected morphology within the image region inferred from spatial hashes.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### EXAMPLES

As described herein and in detail below, the present disclosure enables simultaneously and spatially resolved whole transcriptome sequencing of single-cell cultures on general substrates optimized for cell attachment and high-resolution imaging. With the methods provided herein, after appending the spatial hashes, whole transcriptome of single cells can be sequenced in parallel with commonly available platforms without any sacrifice on RNAseq coverage.

### Example 1

Sequencing Cells with In Situ Spatial Hashing

This Example provides an exemplary method and workflow for in situ spatial hashing of single cells.

In general, the provides sequencing cells with in situ spatial hashing, including the steps of (1) Preparing a cell culture; (2) Preparing a hashing substrate; (3) Applying the hashing substrate and releasing hashes; (4) Dissociating cells to create a particulate suspension; (5) Analyzing or sorting the suspension; (6) Sequencing the suspension; and (7) Bioinformatically mapping cell positions using unique spatial hash or hash combinations.

For each of the above method steps, numerous embodiments are contemplated, as set forth below.
Preparing cell culture
   Load cells on a substrate suitable for desired analysis
      Cells can be grown on a culture substrate for cell growth and morphology development
      Cell culture substrate may contain functional groups for cell attachment
      Cell culture substrate may be a bare surface or contain nanowells for compartmentalization
      Cells may be sorted and printed onto the bare surface or into nanowells
      Microcolonies of cells may be deposited instead of single cells to increase sequencing sensitivity, such as for yeast or bacteria
   Phenotypic and multimodal analysis of cells
      Binding of antibodies, nucleic acid probes, etc.
      Imaging, traction force microscopy (TFM), atomic force microscopy (AFM), mass spectrometry (MS), enzyme assays
Preparing a hashing substrate
   Prepare a hashing substrate for hash oligo capture and controllable release.
      Hashing substrate may be modified with functional groups for oligo crosslinking
      Hashing substrate may be arrayed with nanowells as diffusion barriers using photolithography
      Gelation
         Gelation done in nanowells or bare hashing surface as diffusion barriers and reservoir for hash oligos
         Gelation with polymerization, crosslinking, or high viscosity
         Photo, chemical or temperature initiated
   Micropatterning the array with unique hashes or hash combinations at each position
      Hashes may have binding moieties to the hashing substrate
      Hashes may be trapped in nanowells, gels or on functionalized surface
      Hashes may be lipid-modified to directly insert into the cell surface
      Hashes have binding sequences to the existing LMO oligos on the cell surface
      Hashes have binding sequences to the barcoded beads for sequencing
      Hashes may have fluorescent dyes or other functional groups
   Micropatterning the array with multiple chemicals at each position
      Drugs, proteins, dyes, nucleic acid probes are trapped in nanowells, gels or on functionalized surface
      For cell treatment or labeling/sorting
   Analyzing the array substrate
      Confirm accurate micropatterning with imaging, MS, etc
Stamping the hashing substrate and releasing hashes to cell surface
   Insertion of LMO oligos into the cell surface
      LMOs may be added to cells before or during stamping with the hashing substrate
      LMOs may have different lipid groups.
      Two (anchor and co-anchor) or more lipid types of LMOs are added to cell surface to stabilize the insertion of LMO oligos
      LMO oligos may contain sequences with spatial information or binding sequences for hash oligos
   Gelation.
      Gelation may be done for cell microcolonies to preserve their integrity and increase sequencing sensitivities
      Gelation with polymerization, crosslinking, or high viscosity
      Photo, chemical or temperature initiated
   Stamping the hashing substrate to the cell surface
   Release hash oligos onto cell surface
      Controllable release mechanisms may be thermal melting, photo-mediated cleavage, enzymatic cleavage, pH, or oxidation-reduction reactions
      Released hashes may bind to LMO-oligos on the cell surface
      Released hashes may be lipid-modified and directly insert into the cell surface
   Diffusion gradient of hashes on the cell surface
      Model diffusion gradient to interpolate and triangulate cells between deposited positions of hashes
   Analyzing the spatially hashed cells.
      Imaging, MS, etc
Stamping the hashing substrate and releasing hashes to cell surface Spatial hashing can also be done without the hashing substrate
   Cell fixation
      DSP, methanol, PFA, glyoxal, etc
   LMO insertion
   Hash printing
      More than one spatial oligos can be printed into each nanowell with a predefined pattern
Dissociate cells to create a suspension
   Mechanical disturbance or enzymatic digestion of cells or gel chunks into a particulate suspension
Analyzing and processing the cell suspension
   Labeling the suspension.
      Antibodies, nucleic acid probes, etc
   Analyzing or sorting desired fractions of the suspension
      Fluorescent hashes at specific locations
      Additional labels for cells
      Flow cytometry, imaging, mass spectrometry to analyze or isolate cells
   Dissolving or reversing crosslinks for gel chunks
   Barcoding the suspension
      Accepts disaggregated cells or gel chunks
      Compartmentalization such as droplets or wells
      Adds barcode sequence to disaggregated cell nucleic acids, hashes, antibody-DNA conjugate, etc, to enable single chunk sequencing using “single cell” approaches.
Sequencing and analyzing the suspension
   Link sequencing readouts through barcodes
   Infer spatial position from the set of barcode group associated spatial hashes
      Uses co-occurrence of a unique hash or hash combinations within each barcode group to assign spatial position for the cell
      Modeling the diffusion kinetics can enhance spatial resolution
   Multimodal data integration.
Integration with imaging, AFM, TFM, MS, FACS, Ab-seq, DNA-seq, etc.

### Example 2

### High-Throughput Integration of Single-Cell Morphology and Transcriptome for Individual Neurons

The present Example provides an embodiment that integrates single cell morphology and RNAseq data with stamped spatial hashes. Recent studies have linked cell morphology to multiple cellular processes, such as cell cycle, drug response, gene expression and metastatic potential. For example, neuronal morphology and connectivity indicate the distinct computations each neuronal circuit can perform in the nervous system. Nevertheless, how the underlying variations in gene expression determine the differentiation of neuronal subtypes and morphology remain elusive, and therefore is a subject of on-going research (Que, L., et al. Nat Commun. 2021. 12, 108; Fuzik, J., et al. Nat Biotechnol. 2016. 34, 175-183.). No high-throughput method exists to simultaneously investigate the morphology and transcriptome profiles of the same neuron. Current spatial transcriptomics technologies, such as 10X Visium, XYZeq, Slide-seq, MERFISH, and seqFISH are not suitable, since they are limited by long imaging time, processing difficulties, and reliance on fixed tissues (Rodriques, S. G., et al. Science. 2019. 363 (6434), 1463-1467.; Chen, K. H., et al. Science. 2015. 348 (6233); Shah, S., et al. 2016. Neuron. 92 (2), 342-357.; Lee Y, et al., Science Advances. 2021. 21; 7 (17): eabg4755). Other single-cell technologies such as Takara's ICELL8 (Tirier, S. M., et al. Scientific reports. 2019. 9, 12367) are not compatible with long-term cell culture with adherent cells. Therefore, researchers often need to pick cells or neurons in low throughput, such as with CellCelector or Patch-seq (glass pipettes), for morphological and RNAseq investigations (Wu, P. H., et al., Science Advances, 2020. 6 (4): p. eaaw6938.; Liao M C, et al. J Neurosci. 2016 Feb. 3; 36 (5): 1730-46; Que, L., et al. Nat Commun. 2021. 12, 108; Fuzik, J., et al. Nat Biotechnol. 2016. 34, 175-183. Cadwell, C. R. et al. Nat. Biotechnol. 2016. 34, 199-203.). In this embodiment, single-cell transcriptome-morphology mapping is performed using iPS-derived neurons and stamped hashes.

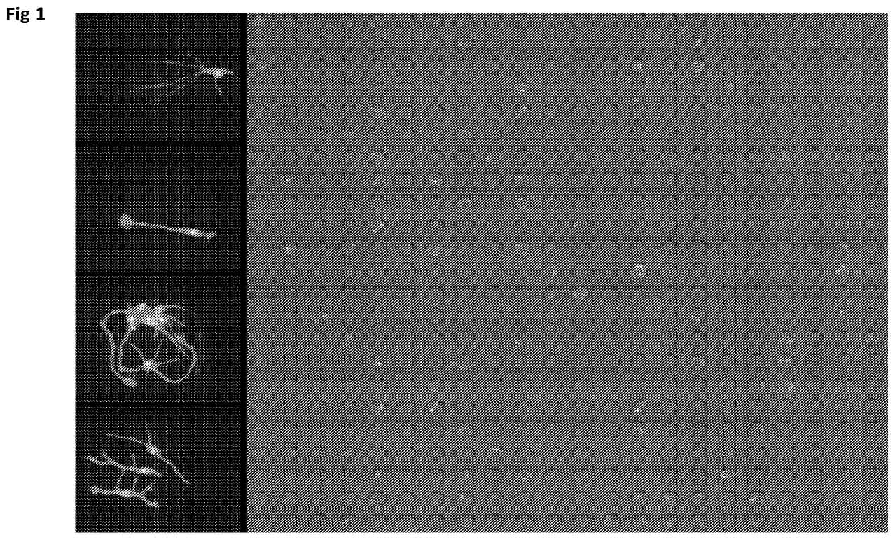

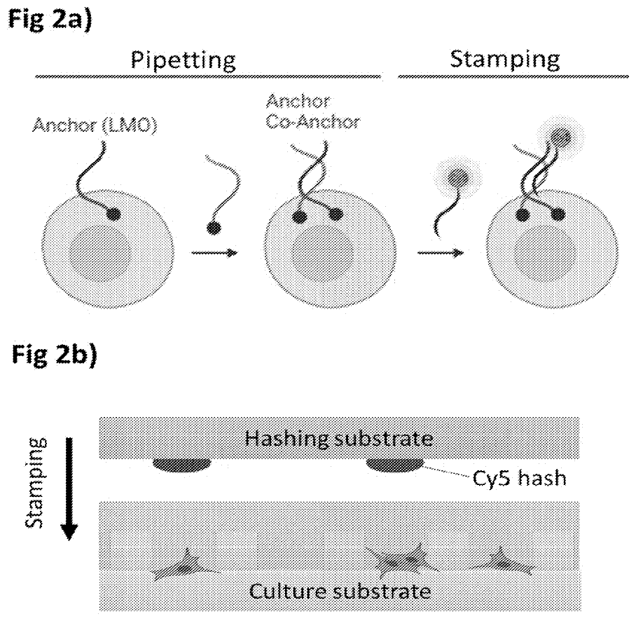

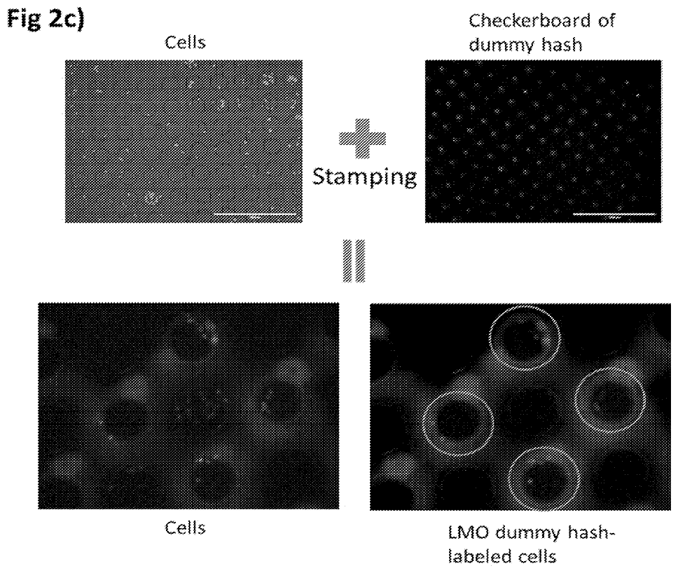

With the present method, single cells can grow and develop morphology indefinitely in isolated nanowells. Thousands to tens of thousands of iPS-derived neurons can be stained and automatically imaged well-by-well with fluorescence microscope (FIG. 1). Whole transcriptome of single cell in each nanowell along with the stamped hashes can be recovered with common next-generation sequencing platforms. Briefly, the workflow includes: 1) Load cells onto a nanowell array of a desirable size. 2) Image cells with fluorescence microscopy. 3) Prepare a hashing substrate that matches the dimension of the nanowell array. 4) Apply the hashing substrate and release the hashes to the nanowell array (FIG. 2). 5) Dissociate cells into buffers with gentle dissociation enzyme. 6) Sequence the suspension with common single-cell platforms, such as 10X Chromium or Fluent BioSciences' PIPseq, etc. 7) Bioinformatically map RNAseq data from cells to nanowells using unique hash combinations.

To facilitate method development, Cy5-labeled dummy hashes were prepared for easy visualization of cell hashing without sequencing (FIG. 2). To estimate the crosstalk of hashes between nanowells, the Cy5 hash was printed on the hashing substrate with checkerboard pattern. After stamping, only cells in nanowells with direct contact with Cy5 hash are expected to be fluorescent in the red channel. A s shown in FIG. 2, cells in the hashed nanowells (blue circles) show strong red fluorescence, while those in the unhashed nanowells (the center nanowell) show little to no red fluorescence, indicating effective hash separation between nanowells.

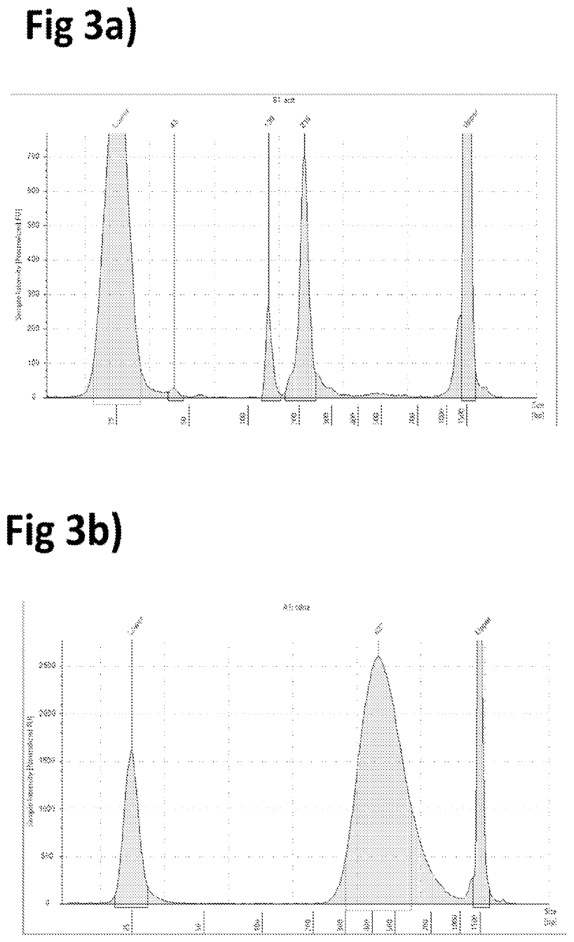

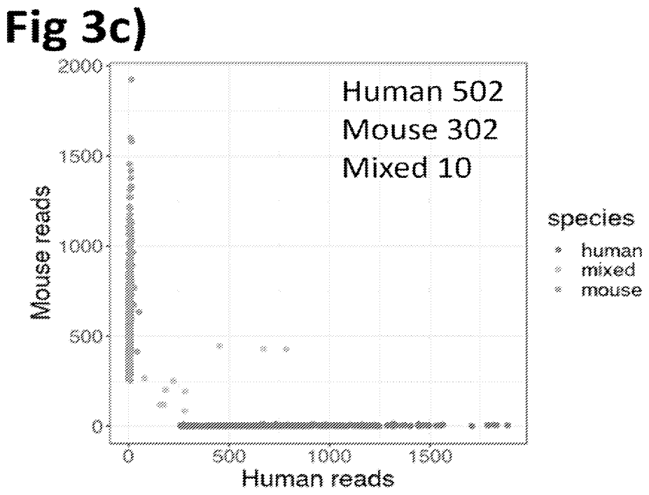

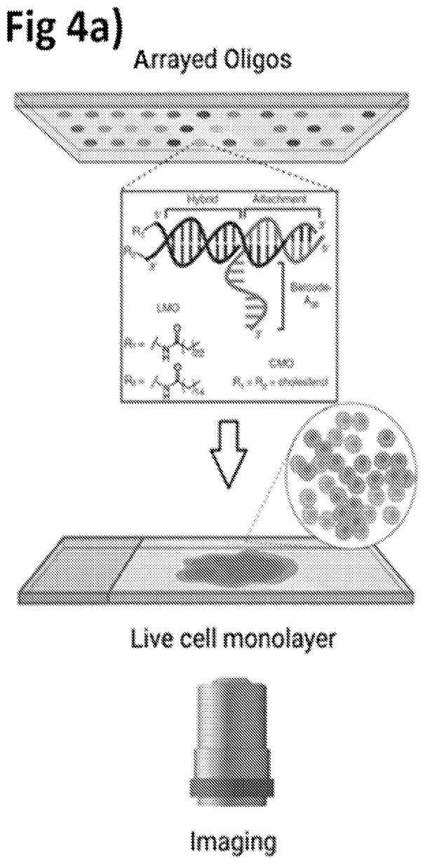

Next, to ensure effective cell recovery and disaggregation for subsequent sequencing, human (HEK293) and mouse (3T3) cells were cultured in the nanowell array. As shown in FIG. 3, both cDNA and spatial hashes were successfully recovered with standard library prep steps. The two-species barnyard plot of the cDNA fraction also show well-separated transcripts from either species, indicating that the cells remain intact throughout the workflow since there is little to no mixing of leaked transcripts between individual cells. It also indicates that the co-encapsulation events, the cell multiplets, are rare.

### Example 3

### Paired Spatial Transcriptomics and Force Profiling for Single Cell Analysis

The present Example provides an embodiment that integrates single cell traction forces and RNAseq with stamped spatial hashes. During embryogenesis, it is well understood that mechanical tension and forces play an important role in modulating cell structural and functional development. However, how these forces influence cell fate determination through transcription, cell signaling, and cell organization remains relatively unclear. Prior studies showed that cell geometries modulate local cell-adhesion tension to direct mesoderm specification in early embryogenesis (Muncie, J. M., et al. Developmental Cell. 2020. 55 (6), 679-694.). However, previous research on the relevance of mechanics in embryonic development lacks the genomic information. To understand how the same human embryo derive into distinct cell types under the highly localized force profiles, it is necessary to characterize gene expression in the spatial context for the emergence of genetic heterogeneity. However, most spatial transcriptomics methods including 10X Visium, XYZeq, Slide-seq, MERFISH, and seqFISH are limited by long imaging time, processing difficulties, and reliance on fixed tissues (Rodriques, S. G., et al. Science. 2019. 363 (6434), 1463-1467.; Chen, K. H., et al. Science. 2015. 348 (6233); Shah, S., et al. 2016. Neuron. 92 (2), 342-357.; Lee Y, et al., Science Advances. 2021. 21; 7 (17): eabg4755). For our purpose to study how mechanical tension influence cell fate determination, no current spatial transcriptomics technologies are compatible with functional single cell analysis such as force profiling because they operate on fixed or frozen tissues rather than live cells.

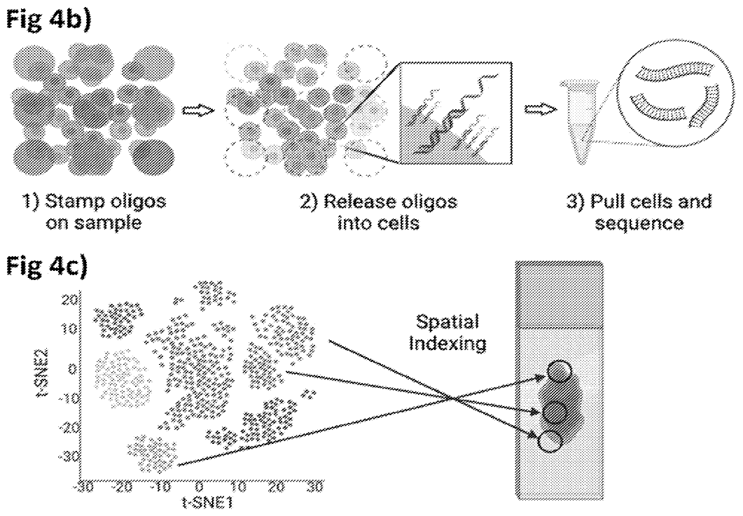

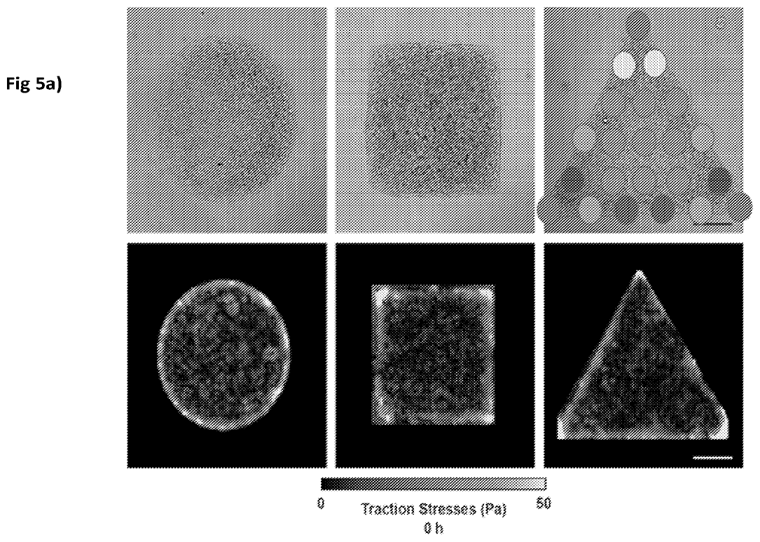

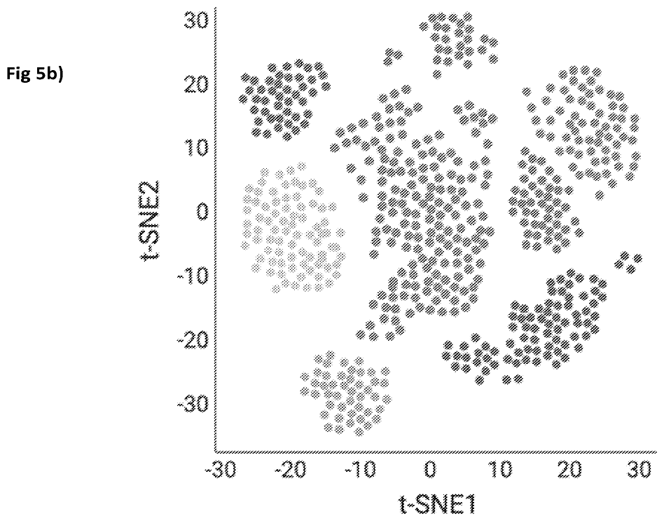

Here, spatial transcriptomics and force profiling (with traction force microscopy, TFM) were paired in the analysis of the same cell sample (FIG. 4). An in-situ spatial transcriptomics method is developed for live cells by hashing the cells with lipid-modified oligonucleotides followed by RNA sequencing. As an application with a goal to study how forces influence early human embryogenesis, a cultured human embryonic stem cell model is used to jointly analyze the cell adhesion tension and spatial transcriptomics data (FIG. 5).

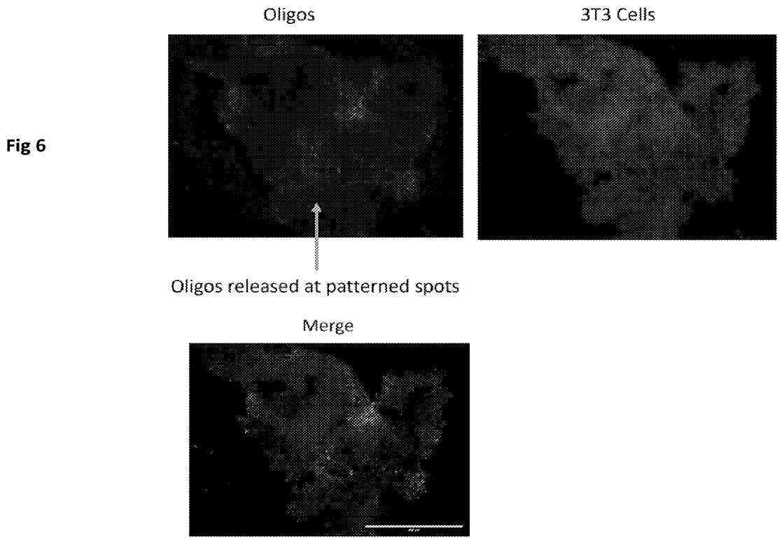

Briefly, the steps include: 1) Load cells onto a patterned substrate for TFM. 2) Image cells with TFM. 3) Prepare a hashing substrate that matches the dimension of the TFM substrate. 4) Apply the hashing substrate and release the hashes to cells (FIG. 6). 5) Dissociate cells into buffers with gentle dissociation enzyme. 6) Sequence the suspension with common single-cell platforms, such as 10X Chromium or Fluent BioSciences' PIPseq, etc. 7) Bioinformatically map cells to force profiles obtained by TFM.

Same as the nanowell format, to facilitate method development, Cy5-labeled dummy hashes were prepared for easy visualization of cell hashing without sequencing. After removal of the excess medium on top of the live cells, the stamped Cy5 hash also shows little crosstalk between printed regions even without separation of nanowells.

### Example 4

### Integrating Transcriptome and Metabolome for Pathway Engineering of Valuable Metabolites

The present Example provides an embodiment that integrates mass spectrometry and RNAseq to engineer an optimal pathway for production of valuable metabolites. Bioproduction enables cheap and efficient synthesis of a large array of compounds under ambient conditions, ranging from direct methane-methanol conversion to complex drugs and natural products with multiple stereocenters that are otherwise difficult, if not impossible, to be chemically synthesized under similar costs and conditions. Achieving high biosynthesis yields requires optimizing expression level of each pathway enzyme to increase the flux of substrates through the pathway and prevent accumulation of unwanted inhibitory intermediates. However, biosynthesis often requires multiple steps; for example, efficient triacetic acid lactone production by Yarrowia lipolytica involves tuning expression of 5 genes (Kelly A. Markham, et al. PNAS. 2018. 115 (9) 2096-2101), while that of opioid hydrocodone by Saccharomyces cerevisiae, 23 genes (Galanie S, et al. Science. 2015. 349 (6252): 1095-100). Identifying, or “debugging”, pathway bottlenecks thus become increasingly difficult with each additional step as the combinatorial complexity grows rapidly (Young E M et al. Metab Eng. 2018. 48:33-43).

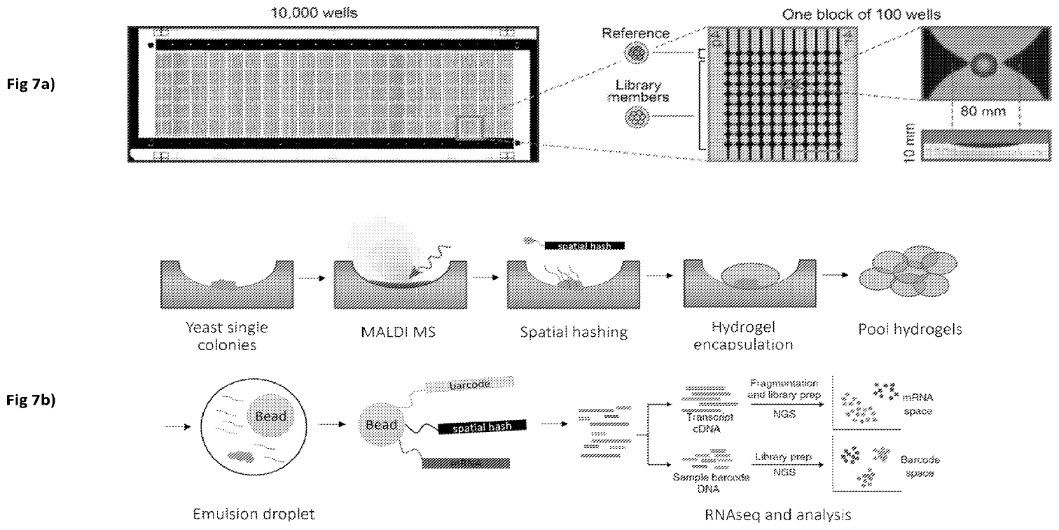

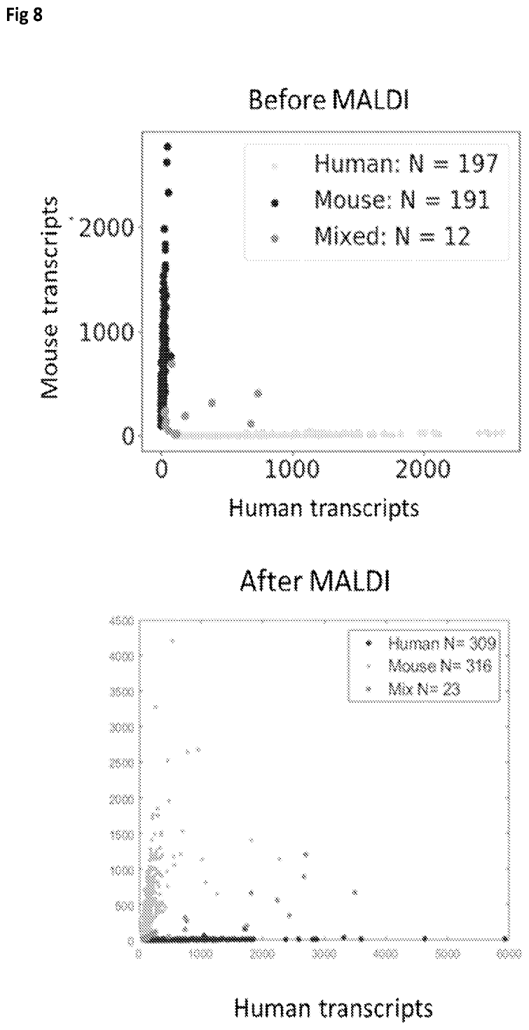

Here, a high-throughput platform was developed to screen a large pathway library of such combinatorial expression levels (FIG. 7). This method is based on the fact that the “soft ionization” nature of MALDI results in relatively mild destruction of the samples being investigated, and we have discovered that both DNA and RNA can be reliably recovered from single cells after MALDI imaging (FIG. 8). Multiple copies of isogenic cells in each microcolony also ensure there are enough metabolites and RNA transcripts for robust multimodal detection.

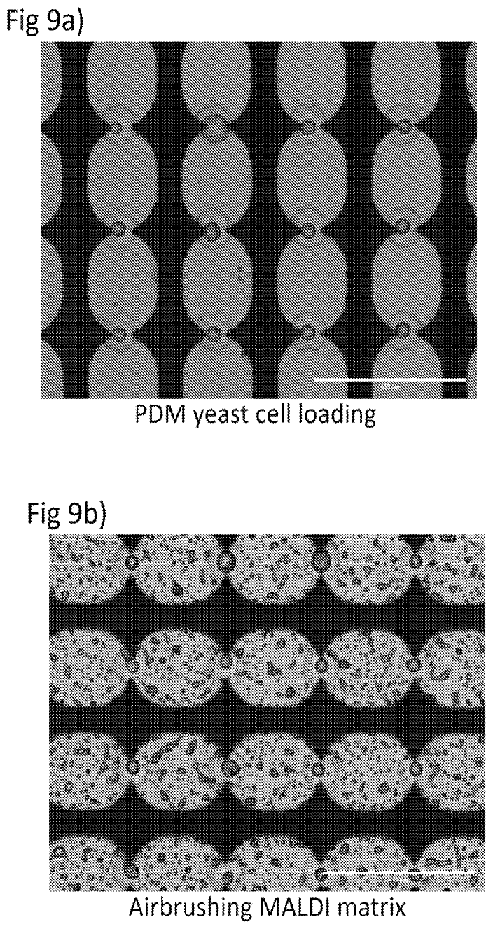

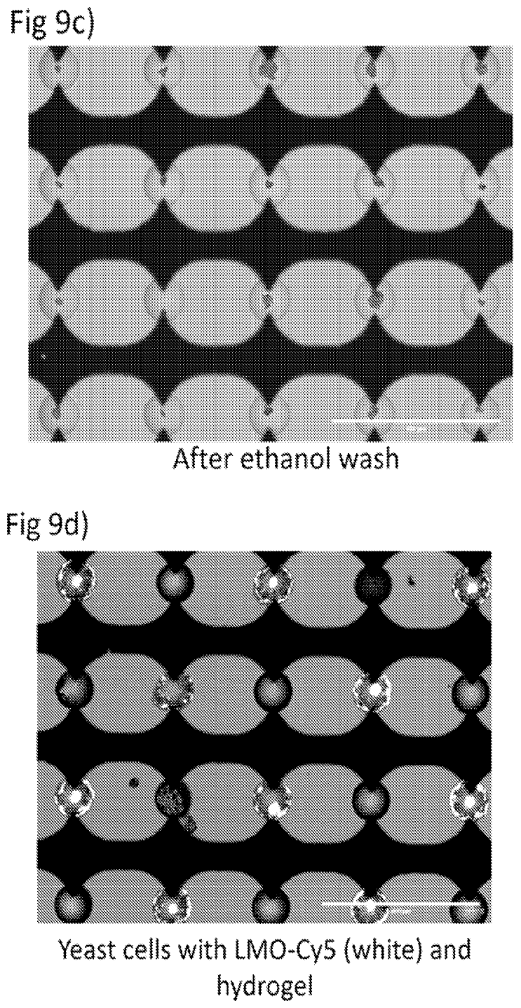

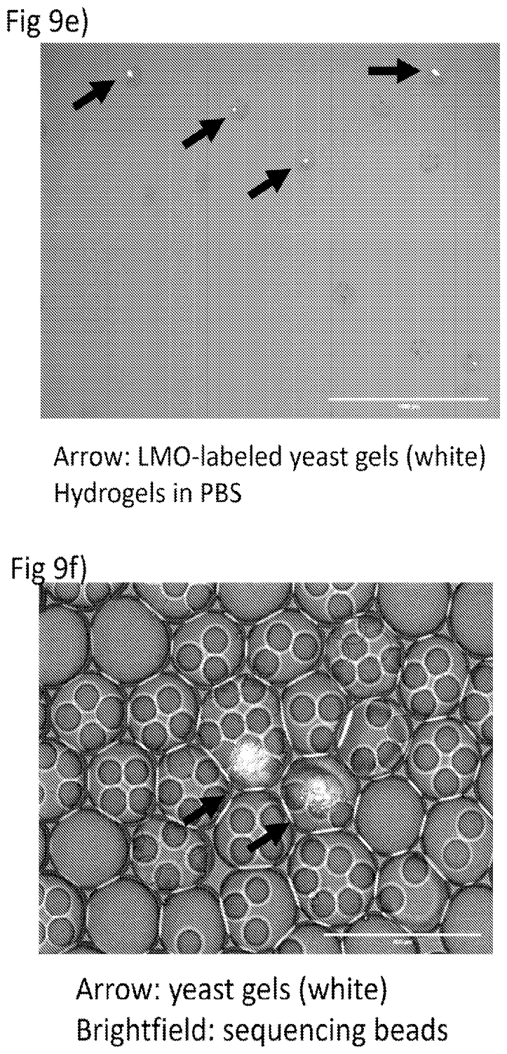

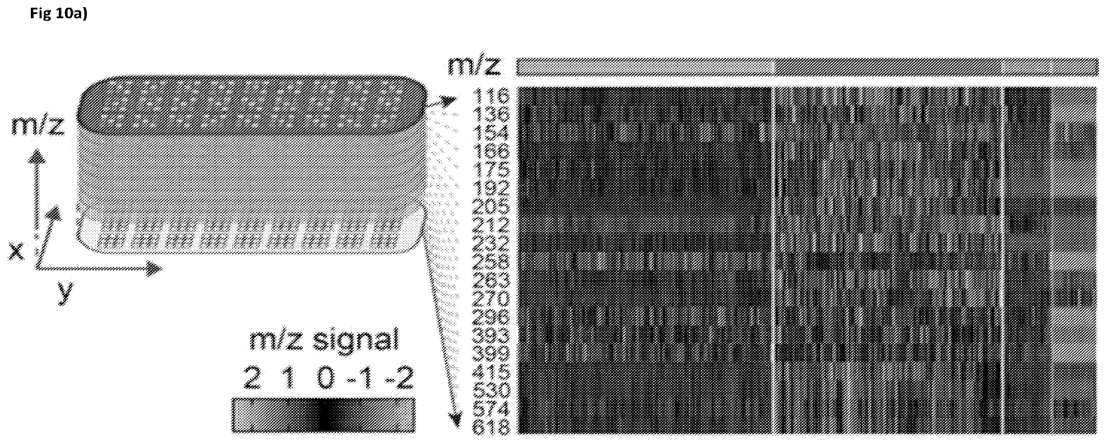

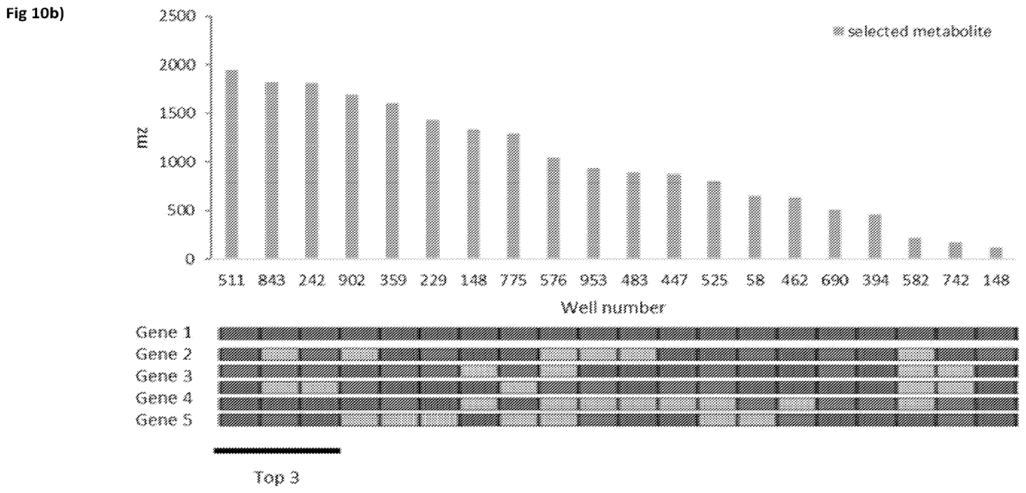

The steps include: 1) Culture single yeast cells in droplets to form isogenic microcolonies. 2) Print microcolony droplets to MALDI substrate with printed droplet microfluidics (PDM) (FIG. 9a). 3) Apply MALDI matrix and measure metabolic production with MALDI imaging (FIG. 9b). 4) Wash the microcolonies (FIG. 9c) and label them with spatial hashes by either stamping the hashing substrate or printing the hashing oligos directly to yeast. 5) Gelled the microcolonies to preserve integrity (FIG. 9d). 6) Resuspend microcolony gel chunks into buffers (FIG. 9e) (similar to our workflow described in Liu, Leqian et al. Lab on a chip. 2019. 19, 10). 7) Encapsulate and dissolve the gel chunks in droplets for single chunk sequencing (FIG. 9f). 8) Bioinformatically map microcolonies to nanowells imaged by MALDI using unique hash combinations (FIG. 10).

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

What is claimed is:

1. A method of determining the sequence of at least one nucleic acid from a single cell, said method comprising the steps of:
(a) preparing a cell culture substrate comprising a plurality of single cells or micro-colonies produced from a single cell;
(b) preparing a labelling substrate, wherein said labelling substrate comprises oligonucleotides comprising spatial barcodes;
(c) labelling single cells or micro-colonies of (a) with labelling substrates of (b);
(d) preparing a suspension comprising labeled single cells or micro-colonies; and
(e) determining the sequence of at least one nucleic acid from at least one labeled, single cell from the suspension.

2. The method of claim 1, wherein the cell culture substrate comprises a surface or a series of wells capable of compartmentalizing micro-colonies or single cells.

3. The method of claim 2, wherein micro-colonies or single cells are deposited onto a surface.

4. The method of claim 2, wherein micro-colonies or single cells are deposited into nano-wells.

5. The method of any of the preceding claims, wherein said method further comprises the step of determining at least one phenotypic or morphologic measurement of the single cells after step (a) and prior to step (c).

6. The method of claim 5, wherein said measurement is selected from the group consisting of imaging, traction force microscopy (TFM), atomic force microscopy (AFM), mass spectrometry (MS), and enzyme assays.

7. The method of any of the preceding claims, wherein the labelling substrate comprises a lipid-modified oligonucleotide (LMO).

8. The method of any of the preceding claims, wherein the labelling substrate is embedded within a gel scaffold.

9. The method of any of the preceding claims, wherein the labelling substrate is deposited onto a surface.

10. The method of claim 9, wherein the surface is selected from the group consisting of an array, a slide, a PDMS slab, a layer of patterned or unpatterned hydrogels, a SU8-coated slide, and a membrane sheet.

11. The method of claim 9 or 10, wherein the labelling in step (c) is carried out under conditions that allow labelling substrates to contact single cells or micro-colonies.

12. The method of claim 11, wherein said conditions comprise inducing release of labelling substrates from the surface.

13. The method of any of the preceding claims, wherein the suspension of (d) is prepared by disassociating and collecting the labeled single cell or micro-colonies.

14. The method of any of the preceding claims, wherein the at least one phenotypic or morphologic measurement is correlated with sequencing information of step (e).
